(19) United States
(12) Patent Application Publication
(10) Pub. No.: US 2026/0026677 A1
(43) Pub. Date: Jan. 29, 2026

(54) ROTATIONAL ASSISTANCE DEVICE FOR SURGICAL INSTRUMENTS

(71) Applicant: Mark Dassel, Cottonwood Heights, UT (US)

(72) Inventor: Mark Dassel, Cottonwood Heights, UT (US)

(21) Appl. No.: 18/881,449

(22) PCT Filed: Jul. 19, 2023
(86) PCT No.: PCT/US2023/070471

§ 371 Date: Jan. 6, 2025

Related U.S. Application Data
(60) Provisional application No. 63/368,920, filed on Jul. 20, 2022.

(51) Int. Cl. A61B 1/00; A61B 1/313

(52) U.S. Cl. CPC A61B 1/00066; A61B 1/00105; A61B 1/00126; A61B 1/00128; A61B 1/3132

(57) ABSTRACT

A surgery system includes a surgical instrument and a rotational assistance device. The surgical instrument includes an elongated element and is configured to be connected to a laparoscopic camera. The rotational assistance device includes one or more interface components attachable to the surgical instrument. The rotational assistance device further includes an extension element extending from the one or more interface components. A force applied to the extension element can cause rotation of the elongated element of the surgical instrument relative to the laparoscopic camera of the surgical instrument.

### CROSS-REFERENCE TO RELATED APPLICATIONS

This application claims priority to U.S. Provisional Patent Application No. 63/368,920, filed on Jul. 20, 2022, and entitled “ROTATIONAL ASSISTANCE DEVICE FOR SURGICAL INSTRUMENTS”, the entirety of which is incorporated herein by reference.

### BACKGROUND

Surgery involves physical intervention on bodily tissues, organs, and/or systems for diagnostic and/or therapeutic purposes. Surgical procedures are typically regarded as invasive procedures that involve the cutting of bodily tissue. Various surgical techniques have arisen to reduce the invasiveness of surgical procedures and/or the amount of tissue that needs to be cut to access, analyze, and/or treat target bodily structures. Laparoscopic surgery (“laparoscopy”) is one of such surgical techniques and is generally regarded as minimally invasive.

Laparoscopy is performed using small incisions in the abdomen or pelvis of a person and with the aid of a camera, referred to as a laparoscope. A laparoscope extends through a small incision and uses a thin tube through which one or more fiber-optic cables extend toward a scope at the tip of the laparoscope. The scope can include one or more optical components (e.g., lenses) for (i) directing light from the fiber optic cable(s) toward bodily structures (e.g., to illuminate the bodily structures) and (ii) for directing light scattered and/or reflected off of the bodily structures toward the fiber optic cable(s) (e.g., for propagation toward an image sensor to provide images/video of the bodily structures). The scope and fiber optic cable(s) thus operate to illuminate the interior of a patient's body and relay a video and/or image feed of internal organs and/or tissues using a video camera. The patient's abdomen or pelvis can be filled with gas to facilitate mobility of the laparoscope within the patient without causing damage to bodily structures. With the visual assistance of the laparoscope, a medical practitioner can operate other surgical equipment (e.g., inserted through other incisions) to perform diagnostic and/or treatment processes.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

Implementations of the present disclosure extend to rotational assistance devices for surgical instruments.

Some embodiments provide a surgery system that includes a surgical instrument that has an elongated element. The surgical instrument is configured to be connected to a laparoscopic camera. The surgery system also includes a rotational assistance device that has one or more interface components that are selectively attachable to the surgical instrument. Assistance device also includes an extension element extending from at least one of the one or more interface components. The rotational assistance device is configured to cause the elongated element of the surgical instrument to rotate relative to the laparoscopic camera when the one or more interface components are selectively attached to the surgical instrument and when a force is applied to the extension element.

Some embodiments provide a rotational assistance device for connection to at least part of a surgical instrument. The rotational assistance device includes one or more interface components that are attachable to a surgical instrument. The rotational assistance device also includes an extension element connected to at least one of the one or more interface components and configured to extend away from at least part of the surgical instrument when the one or more interface components are attached to the surgical instrument. When the rotational assistance device is attached to the surgical instrument and a force is applied to the extension element, the rotational assistance device is configured to cause an elongated element of the surgical instrument to rotate relative to a laparoscopic camera associated with the surgical instrument.

Some embodiments provide a rotational assistance device for connection to a surgical instrument. The rotational assistance device includes a plurality of interface components. The plurality of interface components includes at least a first interface component and a second interface component. The first interface component and the second interface component are selectively connectable to one another to secure about at least part of a surgical instrument. The rotational assistance device also includes an extension element connected to or integrally formed with at least one of the plurality of interface components. The extension element is configured to extend away from the surgical instrument when the plurality of interface components is secured about at least the part of the surgical instrument. When the rotational assistance device is attached to the surgical instrument and a force is applied to the extension element, the rotational assistance device is configured to cause an elongated element of the surgical instrument to rotate relative to a laparoscopic camera of the surgical instrument.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims or may be learned by the practice of the invention as set forth hereinafter.

### BRIEF DESCRIPTION OF THE DRAWINGS

References will be made to embodiments of the disclosure, examples of which may be illustrated in the accompanying figures. These figures are intended to be illustrative, not limiting. Although the disclosure is generally described in the context of these embodiments, it should be understood that it is not intended to limit the scope of the disclosure to these particular embodiments. Items in the figures are not necessarily drawn to scale.

FIG. 1A illustrates a perspective view of an example surgical instrument on which a rotational assistance device may be implemented.

FIG. 1B illustrates a perspective view of an example rotational assistance device selectively secured to an elongated element of a surgical instrument.

FIG. 2 illustrates a disassembled representation of components of an example rotational assistance device proximate to a surgical instrument.

FIG. 3 illustrates a top view of an example rotational assistance device secured to a surgical instrument.

FIG. 4 illustrates a side view of an example rotational assistance device secured to a surgical instrument.

FIG. 5 illustrates a front view of an example rotational assistance device depicting a force applied to the rotational assistance device which results in a rotation of an elongated element of a surgical instrument relative to a laparoscopic camera.

FIG. 6 illustrates example scope configurations for laparoscopes.

### DETAILED DESCRIPTION

Disclosed embodiments are directed to rotational assistance devices for surgical instruments, such as laparoscopes.

Early laparoscopes included an elongated instrument tubing with a scope configured to illuminate and capture video directly along the longitudinal axis of the instrument tubing. More complex surgeries became possible as laparoscopes were developed with scopes configured to illuminate and capture video at an angle that is angularly offset from the longitudinal axis of the instrument tubing. Some angled-scope laparoscopes are additionally able to rotate about the axis of the instrument tubing (and/or about an axis of the laparoscope laparoscopic camera), allowing the view angle of the laparoscope to be changed within the surgical cavity.

Effectively changing the view angle of rotatable laparoscope devices is associated with many challenges. Conventional rotatable laparoscopes are rotatable by manipulation of the instrument tubing itself or by a light post connected to the instrument tubing (e.g., a light post that receives a light source that emits light for propagation through the fiber optics of the laparoscope). Thus, to rotate a conventional rotatable laparoscope, a practitioner would typically hold a laparoscopic camera with one hand and manipulate the instrument tubing (and/or light post or light source) of the laparoscope with their other hand. Surgeons with particularly long fingers may be able to reach the light post to exert force thereon to facilitate rotation of the laparoscope with the same hand used to hold the laparoscopic camera, but this is an uncommon ability. especially as the surgical workforce becomes more diverse and inclusive.

In some instances, rather than rotate a laparoscope themselves, the practitioner may enlist the assistance of another person (e.g., a surgical technician, assistant, or other practitioner) to facilitate manipulation/rotation of the instrument tubing (and/or light post or light source of the laparoscope). Relying on such methods to change the viewing angle of a rotatable laparoscope can prolong and/or disrupt surgical operations, lead to surgeon frustration, introduce risk associated with miscommunication, and/or introduce other inconveniences.

Implementations of the present disclosure extend to rotational assistance devices for surgical instruments (e.g., laparoscope), which may be used to facilitate the rotation of surgical instruments during surgical procedures.

In some implementations, a rotational assistance device attaches to and extends at least partially about a body of a laparoscope. The rotational assistance device can be constructed of one or more interface components and may include one or more attachment features. When attached to the laparoscope, a portion of the rotational assistance device extends outward from the optical axis of the laparoscope and toward the laparoscope eyepiece and/or laparoscopic camera of the laparoscope. The outward-extending portion may form a fin structure that can be manipulated by a person operating the laparoscope to enable rotation of the laparoscope (along with the rotational assistance device) relative to the laparoscopic camera. Such functionality may enable a user (e.g., a surgeon) to hold and rotate a laparoscope with a single hand (e.g., rather than holding the laparoscopic camera with one hand and using their other hand or the hand of another person to rotate the laparoscope).

Providing single-handed rotation functionality as described herein may allow the user to perform other tasks with their other hand in an uninterrupted manner, which can improve operating room efficiency and/or safety, and, consequently, patient outcomes.

In some implementations, the rotational assistance device additionally and advantageously secures a light-guide cable to the laparoscopic light post, thereby reducing inadvertent disconnection of the light-guide cable during use and/or rotation of the laparoscope. Avoiding inadvertent disconnection of light-guide cables can prevent disruptions to surgical operations, which can further contribute to improved efficiency, safety, and/or patient outcomes. Furthermore, since the light cords produce enough heat to burn through sterile drapes and create patient burns. securing the light cord is a patient safety issue. In particular, this safety issue has gained the focus of many health systems as a major safety initiative because each of these drape burns are considered an O.R. fire, a never-event in the surgical theater.

Although the present disclosure focuses, in at least some respects, on examples where a rotational assistance device is implemented on a laparoscope, a rotational assistance device may be implemented on other types of surgical instruments in accordance with the present disclosure. For example, a rotational assistance device may be implemented on any type of minimally invasive surgical instrument (e.g., graspers/forceps. laparoscopic scissors. electrocautery devices. suturing instruments, staplers, slip appliers, retractors, etc.), open surgical instrument, or even on non-surgical instruments.

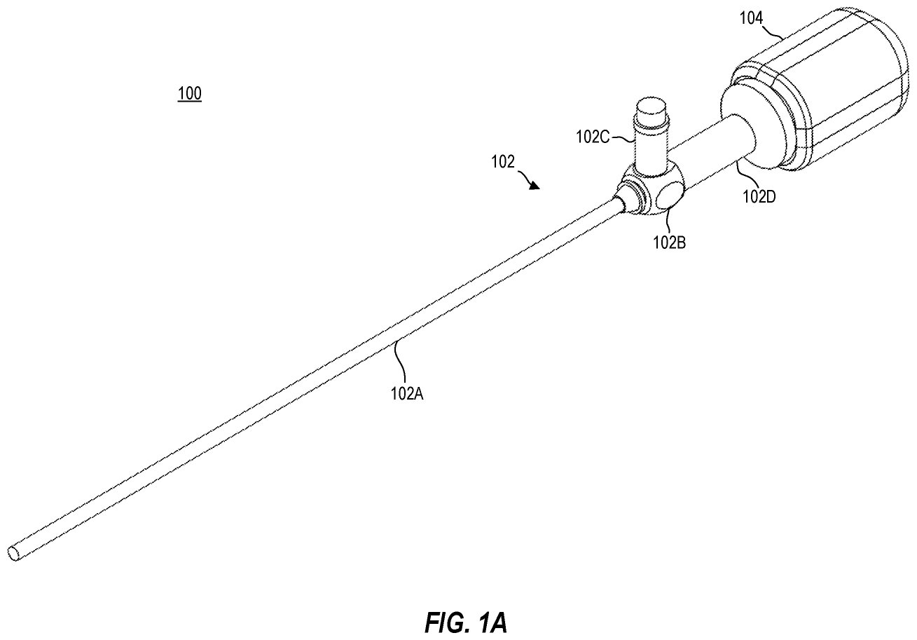

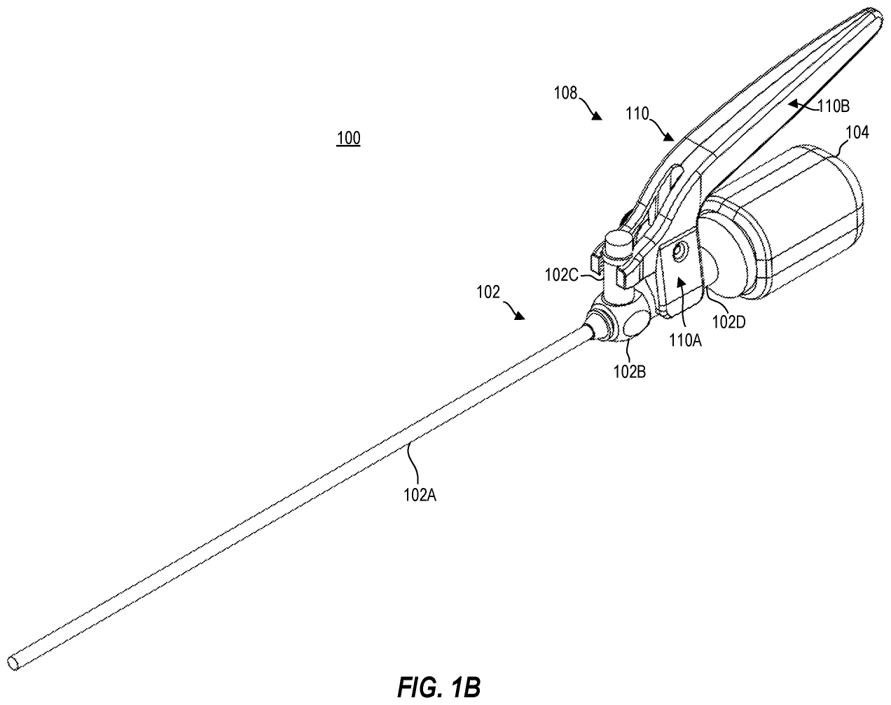

Having just described some of the high-level features and/or advantages associated with the disclosed embodiments, attention will now be directed to FIGS. 1A through 6, which provide supporting illustrations for describing the disclosed embodiments.

FIGS. 1A and 1B illustrate a perspective view of aspects of a surgery system 100, according to implementations of the present disclosure. As shown in FIGS. 1A and 1B, the surgery system 100 includes a surgical instrument 102, a laparoscopic camera 104, and a rotational assistance device 108 (See FIG. 1B). In the illustrated example, the surgical instrument 102 is implemented as a laparoscope and includes an elongated element 102A. a body 102B, a light post 102C, and an eyepiece 102D. As noted herein, other surgical instruments aside from laparoscopes may be implemented in accordance with the present disclosure. Furthermore, the laparoscopic camera 104 shown in FIGS. 1A and 1B comprises a conceptual representation, and other intermediate components may intervene between a laparoscopic camera 104 and a surgical instrument 102.

The laparoscopic camera 104 shown in Figure IA may be regarded as part of the surgical instrument 102 or separate from the surgical instrument 102 (e.g., where the laparoscopic camera is selectively separable from the other components of the surgical instrument 102). In some instances, the laparoscopic camera 104 performs other functions in addition to providing a camera configured to generate images based on light received through fiber optics housed by the elongated element 102A of the surgical instrument 102gripping element. For instance, the laparoscopic camera 104 may comprise or form part of a gripping element for holding the surgical instrument 102.

The surgical instrument 102 of Figure IA can be manipulated by a user via the laparoscopic camera 104. In some instances, the surgical instrument (e.g., the elongated element 102A, the body 102B, the light post 102C, and/or the eyepiece 102D) is configured to rotate relative to the laparoscopic camera 104 (e.g., about an axis extending through the center of the laparoscopic camera 104 and/or the elongated element 102A). Such rotating may enable versatile positioning of the elongated element 102A (and/or components attached thereto) for performance of various procedures. However, as noted above, rotation of such components about the laparoscopic camera 104 under conventional approaches is associated with many challenges. with users often resorting to holding the laparoscopic camera 104 with one hand while using their other hand (or assistance from a third party) to rotate the elongated element 102A relative to the laparoscopic camera 104.

Accordingly, at least some disclosed embodiments provide a rotational assistance device that can enable users to rotate components of a surgical instrument relative to its laparoscopic camera with the same hand used to hold the laparoscopic camera.

FIG. 1B illustrates an example rotational assistance device 108, which may connect to at least part of the surgical instrument 102 and extend toward the laparoscopic camera 104 to enable a user to rotate the surgical instrument 102 relative to the laparoscopic camera 104. In some instances, the rotational assistance device 108 enables users to readily use a single hand to (i) hold the laparoscopic camera 104 and (ii) manipulate the rotational assistance device 108 to effectuate rotation of the elongated element 102A (and/or other components) relative to the laparoscopic camera 104.

In the example of FIG. 1B, the rotational assistance device 108 connects to the surgical instrument 102 such that the rotational assistance device 108 encompasses at least part of the eyepiece 102D and at least part of the light post 102C. One will appreciate, in view of the present disclosure, that a rotational assistance device may at least partially encompass any region or combination of regions of a surgical instrument to provide the rotational assistance functionality described herein. For instance, a rotational assistance device may additionally or alternatively extend over or at least partially encompass the body 102B and/or the elongated element 102A of the surgical instrument 102. By way of further clarification, a rotational assistance device may be regarded as “attached” to at least part of a surgical instrument when the rotational assistance device is secured to an intermediate component that is secured to at least part of the surgical instrument.

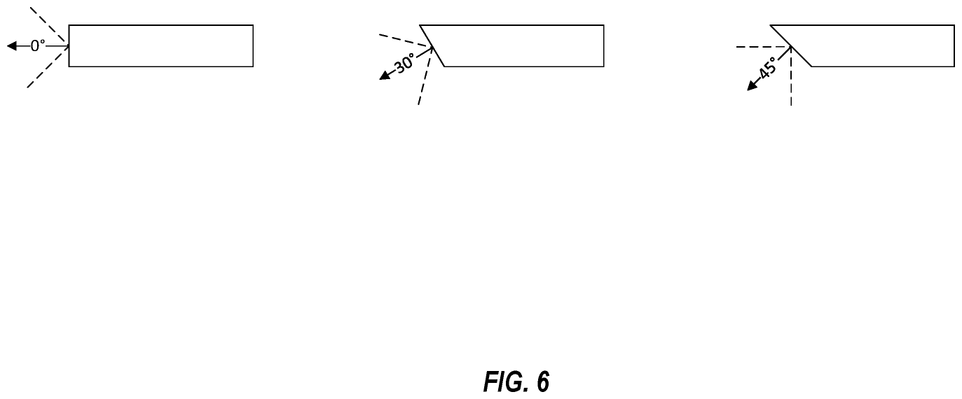

As noted hereinabove, the surgical instrument 102 may comprise various devices/elements connected to regions of the elongated element 102A that extend into patients during surgical procedures (e.g., lenses and/or other optics configured to direct light out of or into the fiber optic(s) within the elongated element 102A). Rotation of the rotational assistance device 108 as discussed above may cause rotation of such devices/elements connected to the elongated element 102A, which can facilitate readily adjustable positioning and/or repositioning of such devices/elements within patients during surgical procedures. For instance, where the surgical instrument 102 is a laparoscope that includes an angled scope for capturing images/video at an angle relative to the main axis of the elongated element 102A (see FIG. 6, which illustrates examples of a non-angled scope (left) and angled scopes (middle and right)). rotation of the rotational assistance device 108 may cause rotation of the field of view for capturing the images/video.

The rotational assistance device 108 of FIG. 1B comprises an extension element 110, which extends from the part of the rotational assistance device 108 that connects to the surgical instrument 102 (e.g., to the light post 102C, the eyepiece 102D, the elongated element 102A, and/or the body 102B). The extension element 110 extends from the point(s) of connection with the surgical instrument 102 toward the laparoscopic camera 104 to enable a user to manipulate the extension element 110 (and, consequently, the surgical instrument 102) using the same hand with which the user holds the laparoscopic camera 104.

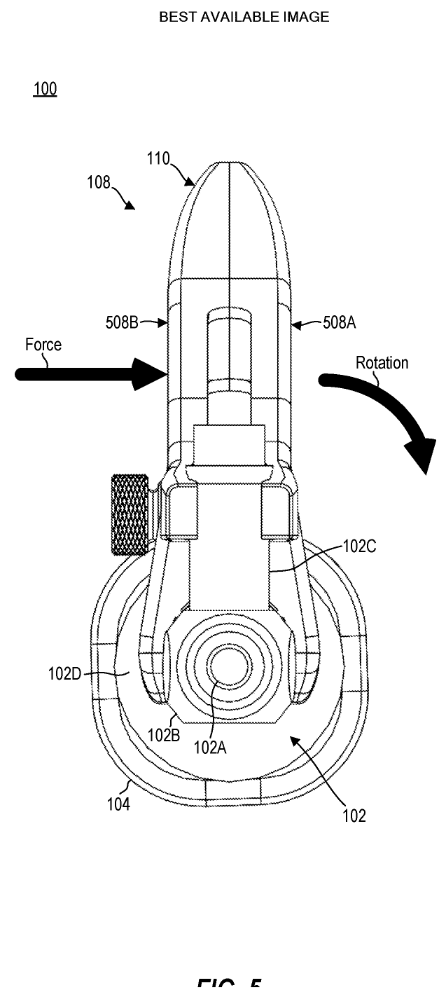

In the example of FIG. 1B, the extension element 110 includes a first region 110A and a second region 110B. The first region 110A of the extension element 110 extends radially away from the centerline (or longitudinal axis) of the elongated element 102A and/or eyepiece 102D. The second region 110B extends from the first region 110A proximally towards the surgical instrument laparoscopic camera 104 (e.g., extending with a directional component that is parallel to the longitudinal axis or centerline of the elongated element 102A and/or eyepiece 102D). A user may manipulate the second region 110B using one or more digits of their hand (e.g., an index finger or thumb) while holding the laparoscopic camera 104 with the rest of their hand. By exerting a lateral force on the second region 110B (and/or first region 110A) of the extension element 110 of the rotational assistance device 108, the user may cause rotation of the second region 110B relative to the laparoscopic camera 104, which, consequently, may cause rotation of the elongated element 102A of the surgical instrument 102 by virtue of the connection between the rotational assistance device 108 and the surgical instrument 102 (see FIG. 5).

As is evident from FIG. 1B, the first region 110A and second region 110B of the extension element 110 may form a fin structure that can be used to rotate the surgical instrument 102 using the same hand in which the surgical instrument 102 is held. The extension element 110 may take on other forms and/or shapes in accordance with the principles described herein. Furthermore, one will appreciate that the designation of a “first region” and/or a “second region” of the extension element is at least somewhat arbitrary and is used for descriptive and/or illustrated purposes only. The extension element 110 may comprise a single unitary part (e.g., where the first and second regions 110A and 110B are integrally formed) or any number of connected parts.

The extension element 110 may extend from the light post 102C of the surgical instrument 102 toward the laparoscopic camera 104 associated with the surgical instrument 102 by a distance that enables users to manipulate the extension element 110 (and therefore the rotational assistance device 108 and the elongated element 102A of the surgical instrument 102) with the same hand used to hold the laparoscopic camera 104. For example, the extension element 110 may extend a distance of about 3 inches to about 6 inches from the light post 102C toward the laparoscopic camera 104 (other distances are within the scope of the present disclosure, such as a distance less than 3 inches or a distance greater than 6 inches). In some instances, the extension element 110 forms about 1 inch to 5 inches of overlap with the laparoscopic camera 104 (along the longitudinal axis of the elongated element 102A) (other overlap distances are within the scope of the present disclosure, such as an overlap distance less than 1 inch or greater than 5 inches).

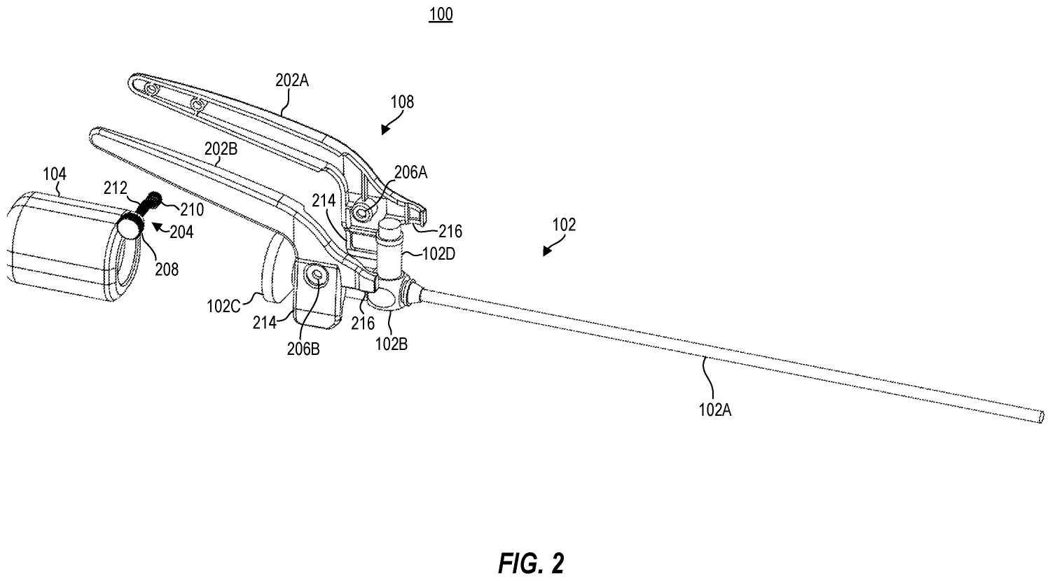

FIG. 1B illustrates an example in which the rotational assistance device 108 is selectively connectable to the surgical instrument 102. The rotational assistance device 108 can connect to the surgical instrument 102 in any suitable manner. FIG. 2 provides a disassembled representation of components of the rotational assistance device 108 proximate to the surgical instrument 102. In the example of FIG. 2, the rotational assistance device 108 includes a first interface component 202A and a second interface component 202B (which may combine to form the extension element 110). The two interface components 202A and 202B are connectable to one another in order to secure to the surgical instrument 102 (i.e., the configuration shown in FIG. 1B).

In the particular example of FIG. 2, the first interface component 202A and second interface component 202B connect together via a threaded member 204 configured to extend through openings 206A and 206B of the first and second interface components 202A and 202B, respectively. The threaded member 204 of FIG. 2 includes a head 208 and is configured to threadedly engage with a nut 210 after extending the threads 212 of the threaded member 204 through the openings 206A and 206B. Rotation of the head 208 relative to the nut 210 after insertion of the threads 212 through the openings 206A and 206B and into initial engagement with the nut 210 can draw the head 208 and the nut 210 toward one another. When the surgical instrument 102 is positioned between the interface components 202A and 202B (e.g., with the eyepiece 102D aligned with eyepiece grips 214 of the interface components 202A and 202B and/or with the light post 102C aligned with light post grips 216 of the interface components 202A and 202B), rotating the head 208 relative to the nut 210 as described above may tighten the interface components 202A and 202B about the surgical instrument 102, thereby securing the rotational assistance device 108 to the surgical instrument 102 (e.g., achieving the configuration shown in FIG. 1B). The head 208 and the nut 210 may be sized to prevent full entry thereof into the respective openings 206A and 206B, thereby achieving the tightening/securing of the rotational assistance device 108 to the surgical instrument 102 as discussed above.

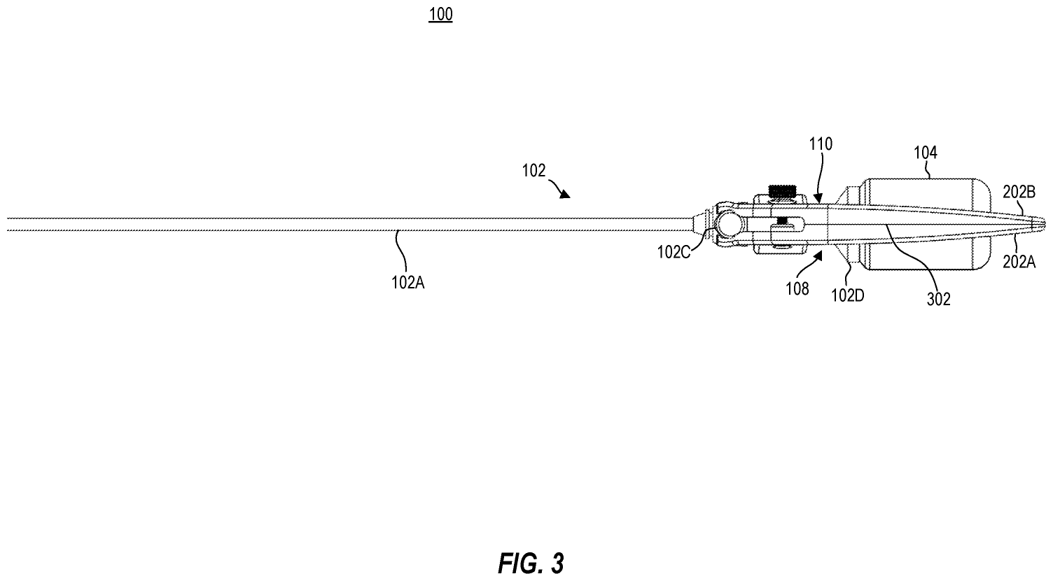

One will appreciate, in view of the present disclosure, that securement of a rotational assistance device 108 to a surgical instrument may be achieved by additional or alternative types of attachment mechanisms (e.g., other than the threaded member/nut example discussed above with reference to FIG. 2). For instance, interface components of a rotational assistance device may comprise detachable annular snap-fit joints (e.g., with plugs and hubs), which can be initially placed on opposing sides of a surgical instrument and brought together such that each plug snaps into a corresponding hub. In some instances, different interface components of a rotational assistance device are joint by a hinge, enabling the different interface components to rotate into position about a surgical instrument prior to securing of the interface components to one another about the surgical instrument. FIG. 3 provides a top view of the rotational assistance device 108 secured to the surgical instrument 102 and indicates potential positioning of a hinge joint 302 between the interface components 202A and 202B.

Although the foregoing examples focus, in at least some respects, on rotational assistance devices that include two interface components that fit about a surgical instrument, a rotational assistance device may include any number of interface components that fit about a surgical instrument, in accordance with the present disclosure. For example, a rotational assistance device may include three interface components that inter-connect with one another and to or about a surgical instrument. As another example, a rotational assistance device may comprise a single unitary interface component that is configured to advance over a front/distal end of the elongated element of the surgical instrument and toward a body. light post, eyepiece, and/or laparoscopic camera of the surgical instrument to secure to the surgical instrument. In yet another example, a rotational assistance device may comprise a single unitary elastic interface component that is deformable to (i) expand a lateral opening of the rotational assistance device to allow the lateral opening to receive an elongated element, body. light post, and/or eye piece of a surgical instrument and (ii) subsequently contract the lateral opening to secure the rotational assistance device about the surgical instrument.

One will appreciate, in view of the present disclosure, that the particular modes of affixation between a rotational assistance device and a surgical instrument provided in the examples discussed hereinabove are provided by way of illustration and/or explanation only and are not limiting of the principles disclosed herein. Any suitable attachment mechanism(s) and/or feature(s) may be utilized to facilitate attachment between a rotational assistance device and a surgical instrument, in accordance with implementations of the present disclosure. For instance, by way of non-limiting example, one or more screws, magnets, hook and loop fasteners, clasps, clamps, clips. zippers, cords/ties, interlocking members, outer housing components, combinations thereof, and/or other components/features may be utilized to attach or connect a rotational assistance device to a surgical instrument. In some implementations. one or more of the attachment mechanisms for connecting a rotational assistance device to a surgical instrument are positioned on or connected to the surgical instrument itself. For instance, an elongated element, body, light post, and/or eyepiece of a surgical instrument may include one or more protrusions (or recesses) configured to engage with corresponding recesses (or protrusions) on a rotational assistance device to facilitate attachment of the rotational assistance device to the surgical instrument.

Attachment mechanisms for securing a rotational assistance device to a surgical instrument may enable a rotational assistance device to secure to a surgical instrument in a manner that avoids or is configured to prevent slipping of the rotational assistance device about the surgical instrument as force is applied to the rotational assistance device (e.g., see FIG. 5). For instance, the attachment mechanism(s) may enable a friction fit between the rotational assistance device and the surgical instrument that is sufficient to maintain the positioning of the rotational assistance device relative to the surgical instrument in response to user forces applied to the rotational assistance device. As another example, one or more mechanical locking members (e.g., protrusions that are configured to engage with corresponding recesses) may be implemented on the surgical instrument and/or rotational assistance device to lock the relative positioning between the rotational assistance device and the surgical instrument as rotational forces are applied to the rotational assistance device.

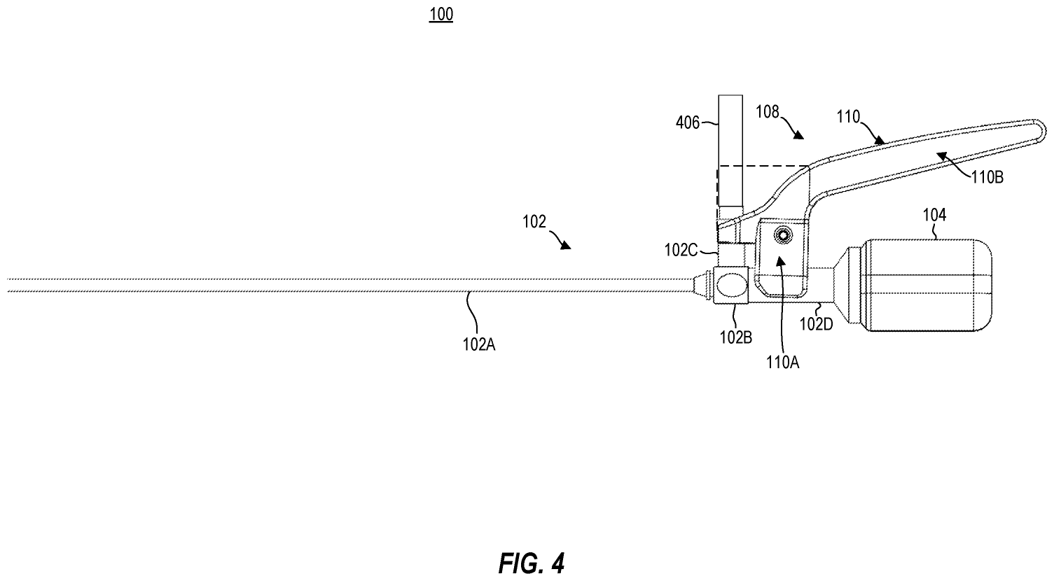

FIG. 4 additionally shows the rotational assistance device 108 at least partially encompassing, gripping, or otherwise engaging with the light post 102C of the surgical instrument 102. The light post 102C of the surgical instrument 102 can be configured to receive a light source 406 (e.g., where light projected from the light source 406 is propagated through fiber optics within the elongated element 102A). For example, the light post 102C may comprise a fiber optic connection port. The light source 406 may comprise one or more fiber optic cables (or other light guide components/devices) that couple to the one or more fiber optic cables of the elongated element 102A. The light source 406 may provide light to one or more fiber optics extending within the elongated element 102A. The one or more fiber optics of the elongated element 102A may propagate the input light toward a scope at a tip of the elongated element 102A (e.g., within a patient during a surgical procedure) to illuminate bodily structures. The elongated element 102A may comprise one or more optics (e.g., lenses) for directing the input light toward the bodily structures. The one or more fiber optics (and/or other fiber optics) of the elongated element 102A may be additionally or alternatively configured to propagate light reflected or scattered off of the bodily structures (as directed by one or more lenses or optics of the scope of the elongated element 102A) toward an image sensor or camera associated with the surgical instrument 102. For instance, the one or more fiber optics may propagate reflected or scattered light toward the eyepiece 102D for perception by an image sensor that may be part of or connected to the laparoscopic camera 104 of the surgical instrument 102.

In some instances, by securing to the light post 102C or fiber optic connection port of the surgical instrument 102, slippage may be prevented in the connection between the rotational assistance device 108 and the surgical instrument 102. For instance. slippage of the rotational assistance device 108 about the surgical instrument 102 during application of force by a user to the extension element 110 of the rotational assistance device 108 is at least partially mitigated by mechanical interfacing between the rotational assistance device 108 (e.g., interface components thereof) and the light post 102C.

In some implementations, when the light source 406 is connected to the light post 102C, the rotational assistance device 108 also encompasses at least a part of the light source 406 (e.g., the light-guide cable), securing the light source 406 to the light post 102C (indicated by dashed lines defining an extended profile of the rotational assistance device 108 in FIG. 4). During a surgical procedure. this securing function, facilitated by the rotational assistance device 108 at least partially encompassing the light source 406 and the light post 102C (and/or other regions of the surgical instrument 102), can prevent inadvertent disconnection of the light source 406 (e.g., light-guide cable) from the light post 102C and can thereby facilitate avoidance of disruptions to surgical procedures and operating room fires, which might otherwise be disrupted by inadvertent disconnection of the light-guide cable from the light post.

FIG. 5 illustrates a front view of the surgery system 100. including the surgical instrument 102, the laparoscopic camera 104, and the rotational assistance device 108. The surgical instrument 102 comprises the elongated element 102A, the body 102B, the light post 102C, and the eyepiece 102D. The rotational assistance device 108 is attached to the surgical instrument 102. FIG. 5 illustrates how a force, as indicated by the arrow labeled “Force,” results in a rotation of the surgical instrument 102 about the laparoscopic camera 104, indicated by the arrow labeled “Rotation.”

By way of illustrative example, referring to FIG. 5. an operator of the surgical system 100 can achieve rotation of the surgical instrument 102 by applying a force upon a surface of the rotational assistance device 108 (e.g., upon an extension element thereof). as indicated in FIG. 5 by the arrow labeled “Force.” The operator can hold the surgical system 100 by firmly grasping the laparoscopic camera 104, with the operator's fingers and palm of their hand supporting the bottom and/or one or more sides of the laparoscopic camera 104. When not applying a force to the rotational assistance device 108, the index finger and/or thumb of the operator can generally rest upon the laparoscopic camera. The operator can apply a force to the rotational assistance device 108 by pressing upon the surfaces 508A and/or 508B of the rotational assistance device 108 (surfaces 508A and 508B being part of the extension element 110 and/or interface components 202A and 202B). The operator of the surgical system 100 may apply the force using one or more digits of the same hand used to hold the laparoscopic camera 104. Alternatively, a hand of the operator (or another entity) not holding the laparoscopic camera 104 can be used to apply the force upon the surfaces 508A and/or 508B to facilitate rotation of the surgical instrument 102 (e.g., the rotational assistance device 108 may provide a more convenient means of producing a rotation of the surgical instrument 102 than relying on rotation of the elongated element 102A and/or other components of the surgical instrument 102).

The applied force results in a rotation of the elongated element 102A of the surgical instrument 102 relative to the laparoscopic camera 104. As noted above, the scope at the tip of the elongated element 102A may be angled (e.g., relative to a main or longitudinal axis of the elongated element 102A and/or laparoscopic camera 104: see FIG. 6) to provide a view angle directed away from the longitudinal axis of the surgical instrument 102. When used in a surgical procedure, the elongated element 102A of the surgical instrument 102 can be inserted into the body of a patient through a small incision. When the scope of the elongated element is so angled and inserted in a patient, a rotation of the elongated element 102A can adjust the field of view of the surgical instrument, permitting an operator of the surgical system 100 to view additional regions of the patient's body. As noted above, when the force resulting in a rotation of the elongated element 102A of the surgical instrument 102 is performed using the hand that holds the laparoscopic camera 104 of the surgical system 100, the rotational assistance device 108 can prevent or mitigate disruptions to surgical operations, thereby contributing to improved efficiency, safety, and/or patient outcomes.

In some implementations, a rotational assistance device is integrally formed (or formed as a unitary part with) a surgical instrument (e.g., with the elongated element, eyepiece, body, and/or light post thereof) to facilitate the single-handed rotation functionality described herein. Under such a configuration. the surgical instrument may be configured to rotate relative to a laparoscopic camera associated with the surgical instrument to modify the orientation of components of the surgical instrument, such as components that are configured to extend into patients during surgical operations. Such rotation may be accomplished by manipulation (e.g., application of force) to the rotational assistance device that is integrally formed with the surgical instrument but still rotatable relative to the laparoscopic camera.

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or regions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

What is claimed is:

1. A surgery system, comprising:
a surgical instrument comprising an elongated element, the surgical instrument being configured to be connected to a laparoscopic camera; and
a rotational assistance device comprising:
one or more interface components that are selectively attachable to the surgical instrument; and
an extension element extending from at least one of the one or more interface components, wherein the rotational assistance device is configured to cause the elongated element of the surgical instrument to rotate relative to the laparoscopic camera when the one or more interface components are selectively attached to the surgical instrument and when a force is applied to the extension element.

2. The surgery system of claim 1, where the surgical instrument comprises a laparoscope.

3. The surgery system of claim 1, wherein the extension element comprises a first region and a second region, wherein the first region extends radially away from a longitudinal axis of the elongated element of the surgical instrument, and wherein the second region extends from the first region proximally toward the laparoscopic camera of the surgical instrument.

4. The surgery system of claim 3, wherein the first region or the second region form a fin structure that, when the rotational assistance device is selectively attached to the surgical instrument, can be used to rotate the surgical instrument using a same hand in which the surgical instrument is held.

5. The surgery system of claim 1, wherein at least one of the one or more interface components and the extension element are integrally formed.

6. The surgery system of claim 1, wherein the rotational assistance device is integrally formed with the surgical instrument.

7. The surgery system of claim 1. wherein the rotational assistance device further comprises one or more attachment mechanisms configured to secure the one or more interface components to the surgical instrument.

8. The surgery system of claim 1, wherein the surgical instrument further comprises an eyepiece and a fiber optic connection port, wherein the rotational assistance device is configured to secure to at least part of the eyepiece and at least part of the fiber optic connection port.

9. A rotational assistance device for connection to at least part of a surgical instrument. the rotational assistance device comprising:
one or more interface components that are attachable to a surgical instrument; and
an extension element connected to at least one of the one or more interface components and configured to extend away from at least part of the surgical instrument when the one or more interface components are attached to the surgical instrument,
wherein, when the rotational assistance device is attached to the surgical instrument and a force is applied to the extension element, the rotational assistance device is configured to cause an elongated element of the surgical instrument to rotate relative to a laparoscopic camera associated with the surgical instrument.

10. The rotational assistance device of claim 9, wherein the extension element comprises a first region and a second region. wherein the first region extends radially away from the elongated element of the surgical instrument.

11. The rotational assistance device of claim 10, wherein the second region extends from the first region proximally toward the laparoscopic camera associated with the surgical instrument.

12. The rotational assistance device of claim 10, wherein the first region or the second region form a fin structure that, when the rotational assistance device is attached to the surgical instrument, can be used to rotate the surgical instrument using a same hand in which the surgical instrument is held.

13. The rotational assistance device of claim 9, further comprising one or more attachment mechanisms configured to secure the rotational assistance device to the surgical instrument.

14. The rotational assistance device of claim 9, where the surgical instrument comprises a laparoscope.

15. The rotational assistance device of claim 14, wherein the elongated element further comprises a fiber optic connection port configured to receive a fiber optic interface, wherein the rotational assistance device is configured to secure to at least part of the fiber optic connection port or the fiber optic interface.

16. A rotational assistance device for connection to a surgical instrument, the rotational assistance device comprising:
a plurality of interface components, the plurality of interface components comprising at least a first interface component and a second interface component, the first interface component and the second interface component being selectively connectable to one another to secure about at least part of a surgical instrument; and
an extension element connected to or integrally formed with at least one of the plurality of interface components. the extension element being configured to extend away from the surgical instrument when the plurality of interface components is secured about at least the part of the surgical instrument, wherein, when the rotational assistance device is attached to the surgical instrument and a force is applied to the extension element, the rotational assistance device is configured to cause an elongated element of the surgical instrument to rotate relative to a laparoscopic camera of the surgical instrument.

17. The rotational assistance device of claim 16, further comprising a hinge connecting the first interface component and the second interface component.

18. The rotational assistance device of claim 16, further comprising one or more attachment mechanisms configured to secure the first interface component to the second interface component about at least the part of the surgical instrument.

19. The rotational assistance device of claim 16, wherein the rotational assistance device is configured to prevent slipping of the rotational assistance device about the surgical instrument during rotation of the rotational assistance device and the elongated element of the surgical instrument relative to the laparoscopic camera.

20. The rotational assistance device of claim 19, wherein the rotational assistance device is configured to prevent slipping by a mechanical locking member.
